(19) United States
(12) Patent Application Publication
(10) Pub. No.: US 2005/0118563 A1
(43) Pub. Date: Jun. 2, 2005

(54) Method for determining embryo quality

(76) Inventors: Geoffrey Sher, Las Vegas, NV (US); Ghanima Maassarani, Las Vegas, NV (US)

Correspondence Address: LATHROP & GAGE LC, 2345 GRAND AVENUE, SUITE 2800, KANSAS CITY, MO, 64108, US

(21) Appl. No.: 10/929,997

(22) Filed: Aug. 30, 2004

Related U.S. Application Data
(60) Provisional application No. 60/498,669, filed on Aug. 28, 2003.

(51) Int. Cl. A01N 1/02; C12N 5/08

(52) U.S. Cl. 435/2; 435/366

(57) ABSTRACT

A method for determining embryo quality involving measuring soluble HLA-G levels present in the embryo culture medium at least 44-46 hours post-fertilization is provided. Culture media and in vitro fertilization programs employing same are also provided.

### RELATED APPLICATIONS

This application is a non-provisional application of provisional application Ser. No. 60/498,669, filed Aug. 28, 2003, the disclosure of which is herein incorporated by reference.

### FIELD OF THE INVENTION

The invention provides a method for determining embryo quality by measuring soluble HLA-G (sHLA-G) levels in the embryo culture media.

### BACKGROUND OF THE INVENTION

A novel gene of non-classical human leukocyte antigen (HLA) class I antigen, HLA-G, was cloned in 1987. This protein is quite different from classical HLA class I antigens (A, B, and C) in that it is almost monomorphic and the site of expression is extremely limited. Soluble human leukocyte antigen (sHLA) class I molecules have been known since 1970, but only recently they have become the subject of intense research because of their presumed importance in the immune response and in the modulation of maternal-fetal immune relationship during pregnancy. HLA-G was first described as a major histocompatibility complex (MHC) class Ib gene exhibiting a very restricted tissue distribution, limited to extra villous cytotrophoblast cells in the placenta, as well as maternal spiral arteries, endothelial cells of fetal vessels in the chorionic villi, in amnion cells, in thymus, and on interferon-γ-stimulated blood monocytes. So far, all of the data demonstrate that the in vivo HLA-G protein expression is restricted to the maternal-fetal interface and thymus. Moreover, the HLA-G molecule is strongly expressed during the first trimester of gestation and then decreases through the remainder, which suggests the role of HLA-G in implantation, as well as a protective function during pregnancy.

U.S. Patent Application 20020015973, filed Feb. 7, 2002, the disclosure of which is herein incorporated by reference, provides a method for determining the potential for successful implantation of an embryo comprising the steps of obtaining a sample of a fluid medium incubating the embryo followed by detecting HLA-G. However, the method disclosed therein does not teach the most effective or appropriate time for measuring sHLA-G levels in the embryo culture media in order to ensure successful embryo transfer.

Thus, it would be a significant contribution to the art to provide a method for determining the quality of embryos for subsequent procedures, including embryo transfer, which measures levels of soluble HLA-G antigens present in the embryo culture media at least 44-46 hours post-fertilization.

### SUMMARY OF THE INVENTION

The present invention provides methods for determining the quality of embryos for use in subsequent procedures, including transfer to the uterus with in vitro fertilization and embryo transfer (IVF/ET) and Tubal Embryo Transfer (TET), by assessing the soluble levels of HLA-G antigens present in the embryo culture media at least 44-46 hours post-fertilization.

### DETAILED DESCRIPTION OF THE INVENTION

The term “antibody” refers to a polypeptide substantially encoded by an immunoglobulin gene or immunoglobulin genes, or fragments thereof. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The term “embryo quality” is defined as a quality indicative of embryos being competent for use in subsequent procedures, including embryo transfer, such as in vitro fertilization, implantation, short-term storage, and long term storage, including cryopreservation. Short term storage may be defined as storage of from about 3 days to about 5 years. Long term storage may be further defined as storage for longer than about 5 years to storage for an indefinite period of time.

The term “HLA-G” refers to human leukocyte antigen G and unless otherwise stated includes both the soluble and insoluble forms. The term may in appropriate context refer to either the antigen or the genetic locus.

The term “immunoassay” is an analysis or methodology that utilizes an antibody to specifically bind an analyte. The immunoassay is characterized by the use of specific binding properties of at least one particular antibody to isolate, target, or quantify the analyte.

The terms “isolated”, “purified”, or “biologically pure” refer to material which is substantially or essentially free from components which normally accompany it as found in its native state.

The term “label” is used in reference to a composition detectable by spectroscopic, photochemical, biochemical, immunochemical, or chemical means. For example, useful labels include ³²P, fluorescent dyes, electron-dense reagents, calorimetric, enzymes, for example, as commonly used in ELISA, biotin, dioxigenin, or haptens and proteins for which antisera or monoclonal antibodies are available can be made detectable.

A critical period of fetal development for survival is that of the early pre-implantation embryo and therefore determining whether HLA-G is expressed during this period is important for understanding its possible role as an embryo protectant. Jurisicova A., et al. (Fertil. Steril. (1996) 65(5):997-1002) reported that it is possible to detect HLA-G heavy chain mRNA in 40% of blastocysts, in some embryos at earlier pre-blastocyst cleavage stages of development (2-4 cell, 5-8 cell, and morula) and in some unfertilized oocytes. In concordance with mRNA data, a similar proportion of embryos stained positive for HLA-G immunohistochemistry (Jurisicova, A., et al. (1996) Proc. Natl. Acad. Sci. USA. 93:161-165). In addition, it was also found that patients who became pregnant and did not have a fetal loss, had a significantly higher proportion of HLA-G positive sibling blastocysts than patients who did not conceive. These studies represented the first report demonstrating the presence of protein and mRNA for the heavy chain of HLA-G, a non-classical class I MHC antigen, and for .beta.2m throughout the whole course of human preimplantation development from the oocyte to blastocyst stages.

Currently, in vitro fertility (IVF) laboratories are able to select pre-embryos only on the basis of their morphology and rate of in vitro cleavage during the first 48 to 72 hours after fertilization. These criteria are useful, but not always good indicators of developmental potential. In most cases, 3 or 4 embryos are chosen based on these relatively crude indicators and then transferred into the uterine cavity. If additional, more stringent pre-embryo selection criteria were available, based on biochemical, genetic or developmental parameters, it would be possible to transfer one or two healthy pre-embryos, which have the highest chance of survival, without exposing patients to the psychological trauma caused by recurrent embryo implantation failure, spontaneous abortions, multiple IVF trials or the risk of multiple pregnancy. Therefore, a more predictive test for successful implantation would be invaluable.

The method of the invention employs a measurement of soluble HLA-G levels present in the embryo culture medium at least 44-46 hours post fertilization. The suitable time for measuring these soluble HLA-G levels may range from at least about 44-46 hours post-fertilization to at least about 144 hours post-fertilization. Measurements may also be taken at times in between these values, and may include measurements of soluble HLA-G levels at 67, 72, 84, and 96 hours post fertilization. Currently, the only available method by which HLA-G can be measured accurately is by the ELISA method, which is time consuming and lacks standardization. Flow cytometric analysis is much less time consuming and, with the establishment of a standard curve, would offer a more rapid and precise method for measuring the concentration of HLA-G in the media. Presently, using ELISA, the concentration of HLA-G has been established in the media surrounding 44-72 hours post-fertilization embryos, which is typically in the range of between about 0.150 and 0.300 OD at 450 nanometers.

In addition, the embryos are evaluated using “Graduated Embryo Scoring (GES). The GES system evaluates embryos during the first 72 hours following fertilization. Each embryo is scored out of a maximum of 100 points. Embryos with a GES score of >70 have the highest chance of developing into viable blastocysts that following embryo transfer (ET) will subsequently implant into the uterine lining (or endometrium) and produce a viable pregnancy. GES thus establishes a sound basis for advising patients with regard to selecting embryos for ET. GES is further discussed herein below in Example 1.

The method according to the invention may optionally comprise the step of measuring HLA-G by comparing the quantity of label detected in the embryo culture media with an HLA-G standard. The sHLA-G employed as a standard may be prepared from the human gestational choriocarcinoma cell line, JEG-3, or the soluble HLA-G molecules may be purified from a human placenta, which may be prepared by employing purified HLA-G from human first trimester placenta tissue. The purification of HLA-G protein has been described in Purification of HLA-G, a Laboratory Manual, (Yie S. M., 1997).

### EXAMPLES

The GES system for evaluating embryo competency based on microscopic development criteria may be applied as provided in Example 1.

### Example 1

### Graduated Embryo Score (GES)

The graduated embryo score (GES) predicts ART outcomebetter than a single day 3 evaluation (i.e., ±72 hours post-fertilization) and achieves results associated with blastocyst transfer from day-3 ET.

Choosing embryos based on serial evaluation of early developmental milestones is superior to an isolated evaluation based on morphology on day 3 and achieves ART outcomes associated with blastocyst transfer from day-3 ET. (Grade A: ≧7cells; <20% fragmentation).

Patients:

Women aged <40 with a normal uterine cavity treated with ART (n=106).

Interventions:

Embryos were graded by GES and by day 3 morphologic characteristics alone prior to ET. Cycle outcomes were compared with embryo grade.

Main Outcome Measures:

On-going gestation and implantation rates.

Results:

Overall on-going gestation and implantation rates were 48% and 26%, respectively. With 1+embryo GES≧70 (n=77), the rates were 62% and 36%, respectively, which were significantly higher than for those with 0 embryos GES≧70 (n=29). With 1+Grade A embryo (n=102) the rates were 50% and 27%, respectively. —of more than one embryo GES≧70 did not improve the pregnancy rate, but did increase the risk of multiple gestations. A single day 3 evaluation had an extremely low specificity (7%) compared to GES (47%). GES was an excellent predictor of pregnancy and implantation rates from blastocyst transfer. Day of transfer did not affect pregnancy rates, although implantation was higher from day 5-ET than from day 3-ET, since fewer embros were transferred.

Conclusions:

Transfer of one or more embryo GES≧70 predicts pregnancy and implantation rates better than a single morphologic evaluation on day 3 and achieves ART outcomes associated with blastocyst transfer from day-3 ET, making extended culture unnecessary for most patients.

Materials and Methods

During the study period, 313 embryos were produced by women under age 40 and were transferred into 106 normal uterine cavities. All patients had medical indications for IVF and were stimulated with recombinant human FSH (Follistim, Organon Inc., West Orange, N.J.) after pituitary down-regulation with GnRHa (Lupron, TAP Pharmaceuticals, Inc, Lake Forest, Ill.) in a long protocol. Follicular development was monitored with serial vaginal ultrasound and serum Estradiol concentrations. Ovulation was triggered with hCG 10,000 IU (Profasi, Serono Inc, Norwell, Mass.) when two lead follicles measured 18 mm in diameter and at least half of the remainder were 15 mm or more in diameter. Oocytes were retrieved transvaginally under ultrasound guidance 34-36 hours after triggering ovulation. Metaphase II oocytes were inseminated four to six hours after retrieval using ICSI in all patients, as is our standard protocol to reduce the risk of unanticipated fertilization failure. Embryos were cultured individually in 50 μl droplets of P1 (Irvine Scientific, Santa Ana, Calif.) +10% Synthetic Serum Substitute (SSS) under oil in a 5%CO₂, 5%O₂, 90%N₂ environment at 37° C. in 95% humidity until day 3 of culture.

Embryos were evaluated by GES on day 1, 2 and 3 of culture and by morphologic appearance (cell number, % fragmentation) on day 3 of culture alone. The GES system and its derivation have been previously described in detail (Table 1). Briefly, GES is the sum of three, weighted, interval evaluations of early developmental milestones, totaling a possible 100 points. Embryos are first evaluated at 16-18 hours post insemination for the presence of nucleolar alignment along the pronuclear axis. Based in part on the work Scott et al. and Tesarik et al., nucleolar alignment was found to be important and was given increased significance in our scoring system. A second evaluation occurs at 25-27 hours post insemination for the presence of regular and symmetrical cleavage, and if so, for percent fragmentation. Early and regular cleavage was noted to be especially important and was given the highest weight. A final evaluation of morphologic characteristics (cell number and fragmentation) occurs 64-67 hours post insemination (day 3 of culture). If an embryo is not cleaved at 25-27 hours, but develops into a Grade A embryo (≧7 cells, <20% fragmentation) on day 3, points for fragmentation are awarded retrospectively.

The highest scoring embryos (mean 3±1) based on GES on day 3 of culture were chosen for transfer. The majority of embryo transfers occurred on day 3 (261 embryos into 83 patients). In our program extended culture is used mainly for patients with prior failures despite having Grade A embryos for transfer and in those whom blastocyst transfer was mandated by their insurance coverage. Day 5-ET patients had the highest GES-scoring embryos on day 3 of those available chosen for transfer. All embryos were transferred atraumatically using a Wallace catheter (Cooper Surgical, Shelton, Conn.) under ultrasound guidance.

Following embryo transfer patients received Progesterone in oil 50 mg a day for luteal support. Serum pregnancy tests were performed 11 and 13 days after egg retrieval. Clinical pregnancy was defined as cardiac activity on vaginal ultrasound performed at 7 to 9 weeks of gestation. Patients doing well at 12 weeks were considered to have an on-going gestation. There was no specific Institutional Review Board approval for this study, since there were no significant effects on management. While we previously advocated transferring only two embryos GES≧70, in this study we chose the number of embryos based on what we felt was optimal for individual patient outcome. The majority of patients in this cohort had three embryos transferred.

The cycle outcomes (on-going gestation and implantation rates) were compared based on: day of transfer, nucleolar alignment, cleavage, embryo grade on day 3 of culture, and GES. Differences between groups were evaluated using Student's t tests. Differences in rates and proportions were evaluated with Chi-Squared Tests and Fisher's Exact Test where appropriate. Significance was set at p<0.05.

Results

Characteristics of the study population are listed in Table 2. The overall ongoing gestation rate was 48% (51/106). Of the 106 patients, 77 (73%) had one or more transferred embryo GES≧70, while 102 (96%) had one or more Grade A embryo transferred. There were initially 26 singletons, 26 sets of twins and 8 sets of triplets. Many of these spontaneously reduced, so that by 12 weeks of gestation there were 41 singletons (80%); eight sets of twins (16%) and two sets of triplets (4%).

Among patients with one or more transferred embryo GES≧70, the on going (>12 weeks) gestation rates was 62% (48/77, which was significantly higher than for the group with no transferred embryos GES≧70 (p<0.001) (Table 2). In comparison, patients with one or more Grade A embryo transferred, had an on-going gestation rate of 50% (51/106), which was not statistically different than for the group with no grade A embryos transferred, due to the small number of patients in that group. No additional predictive value for on-going gestation rate was noted if additional transferred embryos were GES≧70 or Grade A (data not shown).

The multiple gestation rate did rise as the number of embryos transferred scoring GES≧70 increased. No triplets occurred when only one GES≧70 embryo was transferred. With two transferred embryos GES≧70, 8/14 patients initially had twins and 2/14 had triplets. By 12 weeks gestation, several had spontaneously reduced, leaving two ongoing sets of twins and one set of triplets. For patients with three or more transferred embryos scoring GES≧70, 7/21 initially had twins and 4/21 had triplets. At 12 weeks of gestation, there were four ongoing sets of twins and one set of triplets.

Of the 313 transferred embryos, 223 (71%) were GES≧70 and 302 (96%) were Grade A. The overall implantation rate was 26% (82 gestational sacs seen at ultrasound at 6 weeks of gestation/313 transferred embryos). The implantation rate among the group with one or more transferred embryo GES≧70 was 36% (79 sacs/222 embryos), which was significantly higher than 3% (3 sacs/91 transferred embryos) for women with no embryos GES≧70 (p<0.001) (Table 3). GES grading was superior to single morphologic evaluation on day 3 for predicting implantation (p<0.04) (Table 3). Grade A status was not significantly predictive of pregnancy or ongoing gestation, since almost all transferred embryos were Grade A.

One or more cleaved embryo at 25-27 hours was a significant predictor of outcome on its own (Table 3), with an ongoing gestation rate of 61% (37/61). The implantation rate was 36% (63 sacs/175 embryos), compared to 14% (19 sacs/138 embryos) among patients with no cleaved embryos at 25-27 hours post insemination (p<0.001). Nucleolar alignment along the pronuclear axis was not predictive of outcome on its own.

Most patients had embryos transferred on day 3 (83/106) (Table 4). Extended embryo culture was generally reserved for patients with poor quality embryos, repeat failures from day 3 transfer or those mandated by insurance restrictions. There was no difference in pregnancy or implantation rate based on day of transfer alone. Of the 106 patients, 23 had a day 5 transfer. Pregnancy occurred in 9/23 (39%) compared to 4/83 (49%) from day 3-ET. On day 3, 18/23 d5-ET patients had one or more Grade A embryos. Only 12 day 5-ET patients had one or more embryo GES≧70 on day 3 and of these 8 (67%) achieved an on-going gestation compared to 39/64 (60%) among patients having day 3-ET with one or more embryo GES≧70. Couples with one or more embryo GES≧70 had similar pregnancy rates from day 3 or day 5 transfer. The pregnancy rate among day 5-ET patients with no embryos GES≧70 was only 9% (1/11), with a 4% (1/27) implantation rate, despite the embryos having developed into blastocysts. The implantation rate was significantly higher from day 5-ET than from day 3-ET among couples with one or more embryos GES≧70 on day 3 of culture, indicating an additional selective benefit from extended culture among embryos with good early development, which could have implications for reducing the number of embryos transferred.

The statistical values of the two embryo grading systems are compared in Table 5. The positive predictive value (PPV) of an on-going gestation was 62% for the group with 1+embryo graded GES≧70, compared to 50% for the group with 1+Grade A embryo transferred. The sensitivity for the 1+GES≧70 group was 94% compared to 100% for the Grade A group, which is not surprising since only 4% of patients did not have a Grade A embryo. The specificity for the 1+GES≧70 group was 47%, while the specificity for the Grade A group was only 7%. This low specificity means 51/55 (93%) non-pregnant patients had one or more Grade A embryo transferred, while only 29/55 (53%) non-pregnant patients had one or more transferred embryo GES≧70. Cleavage at 25 to 27 hours post insemination was an independent predictor of ongoing gestation, but GES had a higher sensitivity (94% vs. 71%), a higher negative predictive value (90% vs. 69%), and a similar specificity (47% vs. 56%), making it a better overall test for choosing embryos for day 3-ET. The combination of day 5-ET and 1+embryo GES≧70 on day 3 had the highest predictive values and may be especially useful in situations where reducing multiple gestation is an over-riding concern (Table 4).

Discussion:

In this cohort 96% of patients had one or more Grade A embryo transferred, but only 50% conceived an on-going gestation. It is now widely reported that many embryos appearing viable on day 3 will fail to cause a pregnancy. A single morphologic evaluation on day 3 did have 100% sensitivity and 100% negative predictive value, meaning that all patients who conceived had at least one Grade A embryo transferred and none of the patients (n=4/106) without a Grade A embryo conceived. The positive predictive value for an on-going pregnancy was 50%. The problem lies with the 51 (50%) patients who thought they were having good embryos transferred, but who did not conceive.

The specificity of a test is a measure of its false positive rate. In regards to ART success, it could be called the ‘false hope’ rate, since these are the couples who were led to believe their embryos looked good, only to have their hopes dashed when they did not conceive. Of 231 embryos not associated with a gestational sac, 220 (95%) were Grade A. Our data showed that a single evaluation of cell number and morphology on day 3 was unable to adequately distinguish between good and poor quality embryos. It is increasingly clear that additional observations will better identify embryos with the highest chance of implantation.

The introduction of sequential embryo culture media made routine in vitro culture to the blastocyst stage possible. Blastocyst transfer is associated with a high implantation rate, due in a large part, to the fact that 50% or more of phenotypically normal appearing embryos on day 3 will not survive until day 5 and many embryos with arrested development are genetically abnormal. Milki et al. reported many embryos that would have chosen for transfer on day 3 did not correlate with those that subsequently developed into blastocysts. However some embryos with limited developmental potential that may not be able to withstand the stress of extended in vitro culture, may still be robust enough to cause a pregnancy if transferred on day 3.

Blastocyst transfer has been reported to equal or better the on-going pregnancy rate achieved from day 3 transfer, although a recent prospective randomized comparison of day 3 versus day 5 transfer by Levron et al., found day 3 transfer had a better outcome than day 5-ET. This finding is supported by a Cochrane review, which found equivalent outcomes from day 3 or day 5 transfer and recommended routine blastocyst culture be offered with caution since a significant percentage of patients undergoing extended embryo culture will have their cycle cancelled due to complete arrest of embryo development. In our cohort there was no difference in pregnancy rate between day 3-ET and day 5-ET. To minimize the chance of complete developmental arrest, many programs only offer extended culture to patients with a good prognosis for pregnancy in the first place, such as those with four or more 8-cell embryos on day 3. Even with such precautions, some patients with multiple good quality embryos on day 3 will unexpectedly fail to produce any blastocysts on day 5.

A given embryo would be expected to have the same developmental potential on day 3 as on day 5. It is in our ability to distinguish which are the best among a group of high quality candidates that extended embryo culture is potentially helpful. Despite advances in culture technique, it would be arrogant to suggest in vitro conditions could surpass the in vivo tubo-uterine environment and once embryos have been identified for transfer, they should probably be returned to the uterus as soon as possible.

While some assay embryo quality through extended culture, others are focused on timely achievement of early developmental milestones as predictors of implantation potential. Multiple reports have identified early embryo cleavage (24-29 hours after insemination) as a strong positive predictor of outcome and our data support these findings. We found that one or more cleaved embryo for transfer was an independent predictor of outcome and may be a good option for choosing embryos for transfer on day 1-2 of culture.

Several groups report evaluation of pronuclear morphology, (nucleolar alignment, pattern) could also predict outcome from ART, suggesting orderly pronuclear alignment and cleavage are associated with genetically normal embryos. In our analysis nucleolar alignment was not predictive of outcome by itself. Evaluating addition sub-facets of pronuclear morphology, such as perinuclear haloing or nucleolar symmetry, may increase the predictive value.

While rapid embryonic development is important, cleavage speed is not the only factor indicative of normal genetic competence. Ziebe et al. reported transfer of 4-cell embryos on day 2 achieved a better pregnancy rate than those <4 cells, as well as those that had progressed beyond 4-cells. It is our experience that precocious embryo development (>11 cell on day 3) is a negative predictor for blastocyst formation and is supported by the work of Alikani et al. Many, if not most, practitioners would choose an 8-cell embryo for transfer on day 3 over a 10-cell or compacting one. The percentage of fragmentation is another important measure of orderly cell division.

Because multiple factors are involved with embryo development, a single, static observation will invariably miss many embryos which may at first glance appear normal, but which will not result in a live birth. A dynamic, multi-step grading process, such as GES, provides additional opportunities to monitor developmental status. In our original retrospective analysis GES was predictive of blastocyst development and pregnancy following IVF if one or more transferred embryo scored 70 or better. In this study, 60% of day 3-ET and 67% of day 5-ET patients with 1+embryo GES≧70 achieved an ongoing gestation, confirming GES as an excellent predictor of pregnancy from day 3-ET, as well as from blastocyst transfer.

For this study the highest GES-scoring embryos were prospectively selected for transfer. This meant a 7-cell or 9-cell embryo could be chosen over an 8-cell and a Grade II over a Grade I. While 96% of transferred embryos were Grade A, only 71% had a GES≧70. Among the group with 1+embryo GES≧70 (n=77), the on-going gestation rate was 62%, which was higher than for the group with 0 embryos GES≧70. The implantation rate was also significantly higher among patients with 1+embryo GES≧70 (36%), than among those with 0 embryos GES≧70. No multiple gestations occurred in the group with all embryos GES <70, regardless of the number of embryos transferred (max: 5).

Individual embryo culture makes monitoring the developmental progression of specific embryos possible and does not appear to impact embryo quality. In a randomized controlled trial Spyropoulou et al., found no difference in IVF outcome between individual or group embryo culture despite reports indicating group culture improves embryo development. A commitment to monitoring embryos within timed intervals is necessary to successfully implement GES, which may entail embryo evaluation at unusual hours. Timing of evaluations was easily instituted in our laboratory and did not add significant time, cost or labor to the culture process. Repeat removal of the embryos from the incubators also did not appear to affect embryo quality.

The implementation of GES in the program has diminished the potential benefits from extended embryo culture and has made blastocyst transfer unnecessary for most patients. By transferring day 3 embryos selected based on GES, it is possible to avoid the issue of unexpected developmental arrest and achieve a high pregnancy rate with a low rate of multiple gestations. Using GES for serial observations of developmental milestones also increases the specificity of embryo selection. Among our population, 29/55 (53%) non-pregnant patients had one or more transferred embryo GES≧70. While still fairly high, the false positive rate is substantially lower than with a single day 3 evaluation, in which 51/55 (93%) non-pregnant patients had at least one Grade A embryo transferred. The specificity of Day 5-ET with 1+embryo GES≧70 on day 3 was 71% (Table 5).

Based on these findings, serial evaluation of individually-cultured embryos, provides a clearer window on the developmental competence of a given cohort of embryos than a single evaluation on day 1, 2 or 3. Selecting embryos for ET based on GES resulted in similar pregnancy rates from day 3-ET as from day 5-ET, although fewer embryos were transferred on day 5 Additional refinements in GES may further increase its predictive values, which could help to reduce the over-estimation of embryo quality.

TABLE 1
GRADUATED EMBRYO SCORING (GES) OF CLEAVAGE-STAGE
EMBRYOS.

| | Hours after | | |
|---|---|---|---|
| Evaluation | insemination | Developmental milestone | Score |
| 1 | 16-18 | Nucleoli aligned along pronuclear axis | 20 |
| 2 | 25-27 | Cleavage regular and symmetrical | 30 |
| | | Fragmentation¹: | |
| | | Absent | 30 |
| | | <20% | 25 |
| | | >20% | 0 |
| 3 | 64-67 | Cell number and Grade²: | |
| | | 7CI, 8CI, 8CII, 9CI | 20 |
| | | 7CII, 9CII, 10CI, 11CI, Compacting I | 10 |
| Total | | | 100 |
| Score | | | |

¹If the embryo was not cleaved at 25-27 hours, grading of fragmentation should occur at the 64-67 hour evaluation if the embryo reached the 7-cell stage and had <20% fragmentation.
²Grade I = Symmetrical blastomeres and absent fragmentation. Grade II = Slightly uneven blastomeres and <20% fragmentation. Grade III = Uneven blastomeres and >20% fragmentation. Grade A embryos are 7 or more cells with <20% fragmentation.

TABLE 2
Demographic characteristics of 313 embryos derived from 106
women age <40 and transferred into a normal uterus based on a Graduated Embryo
Score (GES) and conventional morphologic evaluation on day 3 of culture.

| | | ≧1 embryo | ≧1 embryo |
|---|---|---|---|
| Characteristic | Total | GES ≧ 70 | Grade A¹ |
| Patients (%) | 106 | 77 (73) | 102 (96) |
| Mean Age (SD) | 33.6 (±4.2) | 33.4 (±4.4) | 33.3 (±4.3) |
| Patients having Day 3-ET | 83 | 65 | 82 |
| Patients having Day 5-ET | 23 | 12 | 20 |
| Transferred embryos (%) | 313 | 222 (71) | 302 (96) |
| Mean embryos transferred | 3.0 | 2.9 | 2.8 |
| Mean embryos transferred on Day 3 | 3.1³ | 3.0³ | 3.1³ |
| Mean embryos transferred on Day 5 | 2.3 | 2.2 | 2.3 |
| On-going pregnancies >12 weeks (%) | 51 (48) | 48 (62) | 51 (50) |
| Singleton (%) | 41 (80) | 38 (79) | 41 (80) |
| Twins (%) | 8 (16) | 8 (17) | 8 (16) |
| Triplets (%) | 2 (4) | 2 (4) | 2 (4) |
| Number of sacs²/Number of embryos | 26% (82/313) | 36% (79/222) | 27% (82/302) |

¹Grade A = ≧7 cells with <20% fragmentation.
²Number of gestational sacs seen on ultrasound at 6 week of gestation.
³p < 0.01 compared to mean embryos transferred on Day 5.

TABLE 3
Distribution of IVF cycle outcomes based whether one or more transferred
embryo achieved the following developmental milestones: Nucleolar
Alignment at 16-18 hours post insemination, Cleavage at 25-27 hours
post insemination, Morphologic evaluation on day 3 of culture
and Graduated Embryo Score (GES).

| | | Nucleolar | | | | | | | |
|---|---|---|---|---|---|---|---|---|---|
| Embryos Transferred | Total | alignment | | Cleavage | | Grade A | | GES ≧ 70 | |
| Embryos with: | | 0 | ≧1 | 0 | ≧1 | 0 | ≧1 | 0 | ≧1 |
| Patients | 106 | 13 | 93 | 45 | 61 | 4 | 102 | 29 | 77 |
| On-going | 51 | 5 | 46 | 14 | 37 | 0 | 51 | 9 | 48 |
| Pregnancy (%) | (48) | (10) | (49) | (31) | (61)¹ | (0) | (50) | (31) | (62)³ |
| Transferred | 313 | 34 | 279 | 138 | 175 | 11 | 302 | 91 | 222 |
| embryos | | | | | | | | | |
| Gestational | 82 | 7 | 75 | 19 | 63 | 0 | 82 | 3 | 79 |
| Sacs seen | | | | | | | | | |
| Implantation | 26% | 21% | 27% | 14% | 36%² | 0% | 27% | 3% | 36%^{3,4} |
| rate | | | | | | | | | |

¹p < 0.003 compared to patients with no cleaved embryos at 25-27 hours post insemination.
²p < 0.001 compared to patients with no cleaved embryos at 25-27 hours post insemination.
³p < 0.001 compared to patients with all transferred embryos GES < 70.
⁴p < 0.04 compared to ≧ 1 transferred embryo Grade A.

TABLE 4
Comparison of IVF outcomes by day of embryo transfer and
Graduated Embryo Score (GES).

| Day of Embryo | | On-going | Implantation Rate |
|---|---|---|---|
| Transfer | n | Pregnancy Rate | (Sacs/Embryos Transferred) |
| Day 3: Total Patients | 83 | 49% (41/83)¹ | 24% (66/261)¹ |
| 1 + embryo GES ≧ 70 | 65 | 60% (39/65)^{2, 3} | 32% (64/198)² |
| 0 embryos GES ≧ 70 | 18 | 11% (2/18) | 3% (2/63) |
| Day 5: Total Patients | 23 | 39% (9/23) | 31% (16/52) |
| 1 + embryo GES ≧ 70 | 12 | 67% (8/12)⁴ | 60% (15/25)^{4, 5} |
| 0 embryos GES ≧ 70 | 11 | 9% (1/11) | 4% (1/27) |

¹Not significant (p > 0.05) compared to Day 5 Total.
²p < 0.01 compared to Day 3 0 ≧ 70.
³Not significant (p > 0.05) compared to Day 5 1 ≧ 70.
⁴p < 0.01 compared to Day 5 0 ≧ 70.
⁵p < 0.01 compared to Day 3 1 ≧ 70.

TABLE 5
Statistical values for predicting pregnancy and on-going gestation
rates from 313 embryos transferred into 106 patients <40 based on whether the
embryos achieved specific developmental milestones: Nucleolar Alignment at 16-18
hours post insemination, Cleavage at 25-27 hours post insemination, Morphologic
evaluation on day 3 of culture, Graduated Embryo Score (GES) and day of embryo transfer.

| One or more | | | | | | |
|---|---|---|---|---|---|---|
| Embryo | Nucleoli | Nucleoli | Grade A¹ | GES ≧ 70 | GES ≧ 70, | GES ≧ 70, |
| Transferred: | Aligned | Cleaved | on day 3 | on day 3 | day 3-ET | day 5-ET |
| Positive Predictive | 49 | 61 | 50 | 62 | 60 | 67 |
| Value (%) | | | | | | |
| Negative Predictive | 23 | 69 | 100 | 90 | 89 | 91 |
| Value (%) | | | | | | |
| Sensitivity (%) | 82 | 71 | 100 | 94 | 95 | 89 |
| Specificity (%) | 6 | 56 | 7 | 47 | 38 | 71 |

¹Grade A = 7 or more cells, <20% fragmentation.

### Example 2

### Purification of Soluble HLA-G Proteins:

Soluble HLA-G proteins were purified using a w6/32 monoclonal antibody (mAb), which recognizes a framework determinant of HLA class I heavy chains associated with human β₂-microglobulin and was used on a sepharose fast flow column to capture sHLA-G molecules from the JEG-3 cell line culture media. There are several commercially specific anti-sHLA-G mabs (Beckman Coulter and Serotec) available, as well as those available from private sources.

Specific sHLA-G ELISA:

A specific sandwich ELISA has been designed to detect sHLA-G. Microtiter plates are coated with specific sHLA-G mAb. After the blocking (usually with bovine serum albumin,) the tested medium/serum/plasma was added. After the incubation, a biotinylated w6/32 mAb was added and after the followed incubation, an enzyme-conjugated streptavidin was added. The reactions are visualized by using an appropriate substrate. Because of lack of standards, so far, the relative concentrations of sHLA-G are estimated only from the absorbency of the yellow product at 492 nm. (Note: if the assay using alkaline phosphatase is employed, the OD is measured at 450 nm; if the assay using peroxidase is employed, the OD is measures at 492 nm.)

In recent work by Fournel et al., different HLA-G mAbs were evaluated for their capability to identify sHLA-G in ELISA. Three of them, 87G, BFL.1, and MEM-G/9, when used as coating Abs together with w6/32 as capture mAb, identified beta2-microglobulin-associated-sHLA-G, but not soluble HLA-B27, in cell culture supernatants from transfected cells. By using these mAbs, sHLA-G was identified in amniotic fluids as well as in culture supernatants of first trimester and term placental explants but not in cord blood. The detection of sHLA-G in embryo culture media suggests that sHLA-G may have a role in evaluating embryo quality and implantation potential in IVF procedures. The authors showed a significant association between sHLA-G antigens and the oocyte cleavage rate measured 48 hours after fertilization.

The human gestational choriocarcinoma cell line, JEG-3, may be used as a source for sHLA-G molecules used as controls in the assay of the present invention.

### Example 3

### Detection of Soluble HLA-G in the Media

The levels of sHLA-G molecule expression in the media surrounding 97 individual embryos of 30 infertile women whose ages ranged between 28-43 years were compared. In each case, at least 2 embryos were selected for transfer 72 hours post fertilization by intracytoplasmic sperm injection (ICSI). Soluble HLA-G expression was compared between morphologically “poor” and “good” quality embryos. All oocytes were fertilized by ICSI and cultured individually in a 50 μl of P-1 media for 60-67 hr. After the embryo transfer (or freezing) the media samples were collected and stored at −30° C. until used. A specific anti-sHLA-G mAb (Beckman Coulter) as coating plate's antibody and w6/32 as capture antibody were used in sandwich ELISA to detect the presence of sHLA-G in each individual media sample. Culture media from choriocarcinoma JEG-3 cell line was utilized as a positive control in order to asses the specificity of the ELISA. The level of sHLA-G expression in each individual sample of P-1 medium was correlated with embryo quality as assessed on day 3 post fertilization using the Graduated Embryo Scoring (GES) System.

A grading for HLA-G expression was established: “Low” (mean OD=<0.120±0.017), “intermediate positive” (mean OD=0.237±0.051) and “strongly positive” (mean OD=0.246±0.045). Embryos were classified into three groups based on such ranges. In Group 1, the culture media of all embryos with a GES of 20-50/100 that were <7 cells cleaved following 72 hrs in culture, showed “low” sHLA-G expression. No pregnancies occurred in this group. Group 2 comprised embryos that had attained 7-9 cells and had a GES of a 70-100, but demonstrated “intermediate positive” sHLA-G expression in the media. No pregnancies occurred in this group. Group 3, embryos comprised those that reached to 7-9 cell stage and each had a GES of 70-100, but in addition showed “strongly positive” sHLA-G expression. Twenty one (21) embryos derived from 6/30 patients (20%) tested “strongly positive” for sHLA-G expression. The clinical pregnancy (ultrasound confirmed) and implantation rates following transfer of these embryos were 84% (5/6) and 43% (9/21) respectively. Twenty-three (23) embryos derived from 8/30 patients (27%) tested “intermediate positive” for sHLA-G expression. The clinical pregnancy (ultrasound confirmed) and implantation rates following embryo transfer of these embryos were 17% (1/6) and 4% (1/23) respectively. Fifty-three (53) embryos derived from 16/30 patients (53%) tested “low” for sHLA-G expression. The clinical pregnancy (ultrasound confirmed) and implantation rates following embryo transfer of these embryos were 0% (0/16) and 0% (0/53) respectively. In addition, there was a strong positive correlation between the amount of sHLA-G in the culture media and the GES as well as the implantation rate per embryo. None of the atretic, arrested, or abnormally looking embryos revealed any sHLA-G expression in the media.

Conclusion:

The presence and concentration of the sHLA-G in the culture medium 72 hrs following fertilization by ICSI could provide a useful indicator measure of subsequent embryo implantation potential.

### References

Alikani, M, Calderon G, Tomkin G, Garrisi J, Kokott M, Cohen J. Cleavage anomalies in early human embryos and survival after prolonged culture in-vitro. Hum Reprod 2000; 15:2634-43.
Almagor M, Bejar C, Kafka I, Yaffe H. Pregnancy rates after the communal growth of preimplantation embryos in vitro. Fertil Steril 1996;66:394-97.
Balaban B, Urman B, Isiklar A, Alatas C, Aksoy S, Mercan R, et al. The effect of pronuclear morphology on embryo quality parameters and blastocyst transfer outcome. Hum Reprod 2001;16:2357-61.
Blake D, Proctor M, Johnson N, Olive D. Cleavage stage versus blastocyst stage embryo transfer in assisted Reproduction (Cochrane Review). Cochrane Database Syst Rev 2002;(2):CD002118.
Blaschitz A, Lenfant F, Mallet V, Hartmann M, Bansusan A, Geraghty D E, Le Bouteiller P, Dohr G: Endothelial cells in chorionic fetal vessels of first trimester placenta express HLA-G. Eur J Immunol 27:3380, 1997.
Bos-Mikich A, Mattos A L, Ferrari A N. Early cleavage of human embryos: an effective method for predicting successful IVF/ICSI outcome. Hum Reprod 2001;16:2658-61.
Chu W, Fant M E, Geraghty D E, Hunt J S: Soluble HLA-G in human placentas: synthesis in trophoblasts and in interferon-gamma-activated macrophages but not placental fibroblasts. Immunology 59:435, 1998.
Crisa L, McMaster M, Ishii J K, Fisher S, Salomon D R; Identification of a thymic epithelial cell subset sharing expression of the class Ib HLA-G molecule with fetal trophoblasts. J Exp Med 186:289, 1997.
Fenwick J, Platteau P, Murdoch A P, Herbert M. Time from insemination to first cleavage predicts developmental competence of human preimplantation embryos in vitro. Hum Reprod 2002;17:407-12.
Fisch J D, Rodriguez H, Ross R, Overby G, Sher G. The Graduated Embryo Score (GES) predicts blastocyst formation and pregnancy rate from cleavage-stage embryos. Hum Reprod 2001; 16:1970-5.
Fournel S, Aguerre-Girr M, Campan A, Salauze L, Berrebi A, Lone Y C, Lenfant F, Bouteiller P: Soluble HLA-G: Purification from eucariotic transfected cells and detection by a specific ELISA. Am J Reprod Immunol 42:22, 1999.
Fournel S, Huc X, Aguerre-Girr M, Solier C, Legros M, Proud-Brethenou C, Moussa M, Chaouat G, Berrebi A, Bensussan A, Lenfant F, Le Bbouteiller P: Comparative reactivity of different HLA-G monoclonal antibodies to soluble HLA-G molecules. Tissue Antigens 55:510, 2000.
Fujii T, Ishitani A, Geraghty D E: A soluble form of the HLA-G antigen is encoded by a mRNA containing intron 4. J Immunol 153:5516, 1994.
Fuzzi B, Rizzo R, Criscuoli L, Noci I, Melchiorri L, Scarselli B, Bencini E, Menicucci A, Baricordi O: HLA-G expression in early embryos is a fundamental prerequisite for the obtainment of pregnancy. Eur J Immunol 32:311, 2002.
Gardner D K, Phil D, Vella P, Lane M, Wagley L, Schlenker T, Schoolcraft W. Culture and transfer of human blastocysts increases implantation rates and reduces the need for multiple embryo transfers. Fertil Steril 1998;69:84-8.
Gianaroli L, Magli M C, Ferraretti A P, Fiorentino A, Garrisi J, Munne S.; Preimplantation genetic diagnosis increases the implantation rate in human in vitro fertilization by avoiding the transfer of chromosomally abnormal embryos. Fertil Steril 1997;68;1128-31.
Haga J A, She J X, Kao K J: Biochemical characterization of 39 kDa class I histocompatibility antigen plasma. A secretable membrane protein derived from transmembrane domain deletion. J Biol Chem 266:3695, 1991.
Hunt J S: HLA and maternal-fetal relationship. Austin, Tex. RG Landes Co, 1996.
Kirszenbaum M, Moreau P, Gluckman E, Dausset J, Carossela E: An alternatively spliced form of HLA-G mRNA in human trophoblasts and evidence for the presence of HLA-G transcript in adult lymphocytes. Proc Natl Acad Sci USA 91:4209,1994.
Le Bouteiller P: HLA class I chromosomal region, genes and products: Facts and questions. Crit Rev Immunol 14:89, 1994.
Le Bouteiller P, Blaschitz A: The functionality of HLA-G is emerging. Immunol Rev 1999.
Levron J, Shulman A, Bider D, Seidman D, Levin T, Dor J. A prospective randomized study comparing day 3 with blastocyst-stage embryo transfer. Fertil Steril 2002;77:1300-01.
Loke Y W, King A: Human Implantation: Cell Biology and Immunlogy. Cambridge, Cambridge University Press, 1995.
Lonergan P, Khatir H, Piumi F, Rieger D. Humblot P, Boland M P. Effect of time interval from insemination to first cleavage on developmental characteristics, sex ratio and pregnancy rate after transfer of bovine embryos. J Reprod Fertil 1999; 117:159-67.
Ludwig M, Schopper B, Al-Hasano S, Diedrich K. Clinical use of a pronuclear stage score following intracytoplasmic sperm injection: impact on pregnancy rates under the conditions of the German embryo protection law. Hum Reprod 2000;15:325-9.
Lundin K, Bergh C, Hardarson T. Early embryo cleavage is a stron indicator of embryo quality in human VF. Hum Reprod 2001;16:2652-7.
Magali M C, Jones G M, Gras L, Gianaroli L, Korman I, Trounson A. Chromosome mosaicism in day 3 aneuploid embryos that develop to morphologically normal blastocysts in vitro. Hum Reprod 2000;15:1781-86.
McMaster M, Zhou Y, Shorter S, Kapasi K, Geraghty D, Lim K, Fisher S: HLA-G isoforms produced by placental cytotrophoblast and found in amniotic fluid are due to unusual glycosilation. J Immunol 160:5922, 1998.
Menicucci A, Noci I, Fuzzi B, Criscuoli L, Baricordi O, Mattiuz P1: Non-classic sHLA class I in human oocyte culture medium. Hum Immunol 60:1057, 1999.
Milki A A, Hinckley M D, Gebhart J, Dasig D, Westphal L, Behr B. Accuracy of day 3 criteria for selecting the best embryos. Fertil Steril 2002;77:1191-5.
Milki A A, Hinckley M D, Fisch J D, Dasig D, Behr B. Comparison of Day 3-ET to Blastocyst-ET in a Similar Patient Population: Fertil Steril 2000;73(1):126-9.
Milki A A, Fisch J D, Behr B.; Two blastocyst transfer has similar pregnancy rates and a decreased multiple gestation rate compared to three blastocyst transfer. Fertil Steril 1999;72:225-8.
Puissant F, Van Rysselberge M, Barlow P, Deweze J, Leroy F. Embryo scoring as a prognostic tool in IVF treatment. Hum Reprod 1987;2:705-8.
Puppo F, Scudetti M, Indiveri F, Ferrone S: Serum HLA class I antigens: markers and modulators of an immune response? Immunol Today, 16:435, 1998.
Rijnders P M, Jansen C A. The predictive value of day 3 embryo morphology regarding blastocyst formation, pregnancy and implantation rate after day 5 transfer following in-vitro fertilization or intracytoplasmic sperm injection. Hum Reprod 1998;13:2869-73.
Shoukir Y, Campana A, Farley T, Sakkas D. Early cleavage of in-vitro fertilized human embryos to the 2-cell stage: a novel indicator of embryo quality and viability. Hum Reprod 1997;12:1531-6.
Scott L A, Smith S. The successful use of pronuclear embryo transfers the day following oocyte retrieval. Hum Reprod 1998;13:1003-13.
Scott L, Alvero R, Leondires M, Miller B. The morphology of human pronuclear embryos is positively related to blastocyst development and implantation. Hum Reprod 2000;15:2394-403.
Spyropoulou I, Karamalegos C, Bolton V N. A prospective randomized study comparing the outcome of in-vitro fertilization and embryo transfer following culture of human embryos individually or in groups before embryo transfer on day 2. Hum Reprod 1999;14:76-9.
Tesarik J, Junca A M, Hazout A, Aubriot F X, Nathan C, Cohen-Bacrie P, Dumont-Hassan M. Embryos with high implantation potential after intracytoplasmic sperm injection can be recognized by a simple, non-invasive examination of pronuclear morphology. Hum Reprod 2000;15:1396-99.
Tsai Y C, Chung M T, Sung Y H, Tsai F, Tsai Y T, Lin L Y. Clinical value of early cleavage embryo. Int J Gynaecol Obstet 2002;76:293-7.
Yang Y, Chu W, Geraghty D E, Hunt S: Expression of HLA-G in human mononuclear phagocytes and selective induction by IFN-gamma. J Immunol 156:4224,1996.
Van Lierop M, Wijnands F, Loke Y, Emmer P, Lukassen H, Braat D, Van der Meer A, Mosselman S, Josten I: Detection of HLA-G by specific sandwich ELISA using monoclonal antibodies G233 and 56B. Mol Hum Reprod 776, August 2002.
Van Rood J J, Van Leeuwan A, Van Santen M C T: Anti-HLA-A2 inhibitor in normal human sera. Nature 226: 336, 1970.
Ziebe S, Petersen K, Lindenberg S, Andersen A G, Gabrielsen A, Andersen A N. Embryo morphology or cleavage stage: how to select the best embryos for transfer after in-vitro fertilization. Hum Reprod 1997; 12:1545-9.
Zollner U, Zollner K P, Hartl G, Dietl J, Steck T. The use of a detailed zygote score after IVF/ICSI to obtain good quality blastocysts: the German experience. Hum Reprod 2002;17:1327-33.

1. A method for determining embryo quality by measuring the levels of soluble HLA-G in the embryo culture medium at least 44-46 hours post fertilization.

2. The method of claim 1 wherein the soluble HLA-G in the embryo culture medium is measured at least 67 hours post fertilization.

3. The method of claim 1 wherein the soluble HLA-G in the embryo culture medium is measured at least 72 hours post fertilization.

4. The method of claim 1 wherein the soluble HLA-G in the embryo culture medium is measured at least 84 hours post fertilization.

5. The method of claim 1 wherein the soluble HLA-G in the embryo culture medium is measured at least 96 hours post fertilization.

6. The method of claim 1 which additionally comprises a step wherein the embryos are evaluated using a graduated embryo score.

7. The method of claim 2 which additionally comprises a step wherein the embryos are evaluated using a graduated embryo score.

8. The method of claim 3 which additionally comprises a step wherein the embryos are evaluated using a graduated embryo score.

9. The method of claim 4 which additionally comprises a step wherein the embryos are evaluated using a graduated embryo score.

10. The method of claim 5 which additionally comprises a step wherein the embryos are evaluated using a graduated embryo score.

11. The method of claim 1 wherein embryo quality is used to determine the potential for successful implantation of an embryo.

12. The method of claim 2 wherein embryo quality is used to determine the potential for successful implantation of an embryo.

13. The method of claim 3 wherein embryo quality is used to determine the potential for successful implantation of an embryo.

14. The method of claim 4 wherein embryo quality is used to determine the potential for successful implantation of an embryo.

15. The method of claim 5 wherein embryo quality is used to determine the potential for successful implantation of an embryo.

16. The method of claim 1 wherein embryo quality is used to determine the potential for successful short term storage of an embryo.

17. The method of claim 2 wherein embryo quality is used to determine the potential for successful short term storage of an embryo.

18. The method of claim 3 wherein embryo quality is used to determine the potential for successful short term storage of an embryo.

19. The method of claim 4 wherein embryo quality is used to determine the potential for successful short term storage of an embryo.

20. The method of claim 5 wherein embryo quality is used to determine the potential for successful short term storage of an embryo.

21. The method of claim 1 wherein embryo quality is used to determine the potential for successful long term storage of an embryo.

22. The method of claim 2 wherein embryo quality is used to determine the potential for successful long term storage of an embryo.

23. The method of claim 3 wherein embryo quality is used to determine the potential for successful long term storage of an embryo.

24. The method of claim 4 wherein embryo quality is used to determine the potential for successful long term storage of an embryo.

25. The method of claim 5 wherein embryo quality is used to determine the potential for successful long term storage of an embryo.

26. The method of claim 21 wherein said long term storage of an embryo involves cryopreservation.

27. The method of claim 22 wherein said long term storage of an embryo involves cryopreservation.

28. The method of claim 23 wherein said long term storage of an embryo involves cryopreservation.

29. The method of claim 24 wherein said long term storage of an embryo involves cryopreservation.

30. The method of claim 25 wherein said long term storage of an embryo involves cryopreservation.

31. The method of claim 1 wherein said levels of soluble HLA-G in the embryo culture medium are from about 0.150 to about 0.300 OD₄₅₀.

32. The method of claim 2 wherein said levels of soluble HLA-G in the embryo culture medium are from about 0.150 to about 0.300 OD₄₅₀.

33. The method of claim 3 wherein said levels of soluble HLA-G in the embryo culture medium are from about 0.150 to about 0.300 OD₄₅₀.

34. The method of claim 4 wherein said levels of soluble HLA-G in the embryo culture medium are from about 0.150 to about 0.300 OD₄₅₀.

35. The method of claim 5 wherein said levels of soluble HLA-G in the embryo culture medium are from about 0.150 to about 0.300 OD₄₅₀.

36. An embryo culture medium comprising an amount of soluble HLA-G from about 0.150 to about 0.300 OD₄₅₀, wherein said medium provides an embryo that is competent for use in subsequent procedures, and wherein said soluble HLA-G level is measured at 44-46 hours post fertilization.

37. The culture medium of claim 36 wherein subsequent procedure is selected from the group consisting of embryo transfer, in vitro fertilization, implantation, short-term storage, long term storage, and cryopreservation.

38. An embryo culture medium comprising an amount of soluble HLA-G from about 0.150 to about 0.300 OD₄₅₀, wherein said medium provides an embryo that is competent for use in subsequent procedures, and wherein said soluble HLA-G level is measured at 67 hours post fertilization.

39. The culture medium of claim 38 wherein subsequent procedure is selected from the group consisting of embryo transfer, in vitro fertilization, implantation, short-term storage, long term storage, and cryopreservation.

40. An embryo culture medium comprising an amount of soluble HLA-G from about 0.150 to about 0.300 OD₄₅₀, wherein said medium provides an embryo that is competent for use in subsequent procedures, and wherein said soluble HLA-G level is measured at 72 hours post fertilization.

41. The culture medium of claim 40 wherein subsequent procedure is selected from the group consisting of embryo transfer, in vitro fertilization, implantation, short-term storage, long term storage, and cryopreservation.

42. An embryo culture medium comprising an amount of soluble HLA-G from about 0.150 to about 0.300 OD₄₅₀, wherein said medium provides an embryo that is competent for use in subsequent procedures, and wherein said soluble HLA-G level is measured at 84 hours post fertilization.

43. The culture medium of claim 42 wherein subsequent procedure is selected from the group consisting of embryo transfer, in vitro fertilization, implantation, short-term storage, long term storage, and cryopreservation.

44. An embryo culture medium comprising an amount of soluble HLA-G from about 0.150 to about 0.300 OD₄₅₀, wherein said medium provides an embryo that is competent for use in subsequent procedures, and wherein said soluble HLA-G level is measured at 96 hours post fertilization.

45. The culture medium of claim 44 wherein subsequent procedure is selected from the group consisting of embryo transfer, in vitro fertilization, implantation, short-term storage, long term storage, and cryopreservation.

46. An in vitro fertilization (WF) program comprising:
contacting a human egg with a human sperm to form an fertilized egg;
growing the resulting embryo in vitro in a chemically defined medium;
measuring the levels of soluble HLA-G in the medium at least 44-46 hours post fertilization; and
transferring the embryo into a compatible human uterus.

47. An IVF program of claim 46 wherein the level of soluble HLA-G is from about 0.150 to about 0.300 OD₄₅₀.

48. An in vitro fertilization (IVF) program comprising:
contacting a human egg with a human sperm to form an fertilized egg;
growing the resulting embryo in vitro in a chemically defined medium;
measuring the levels of soluble HLA-G in the medium at least 67 hours post fertilization; and
transferring the embryo into a compatible human uterus.

49. An IVF program of claim 48 wherein the level of soluble HLA-G is from about 0.150 to about 0.300 OD₄₅₀.

50. An in vitro fertilization (IVF) program comprising:
contacting a human egg with a human sperm to form an fertilized egg;
growing the resulting embryo in vitro in a chemically defined medium;
measuring the levels of soluble HLA-G in the medium at least 72 hours post fertilization; and
transferring the embryo into a compatible human uterus.

51. An IVF program of claim 50 wherein the level of soluble HLA-G is from about 0.150 to about 0.300 OD₄₅₀.

52. An in vitro fertilization (IVF) program comprising:
contacting a human egg with a human sperm to form an fertilized egg;
growing the resulting embryo in vitro in a chemically defined medium;
measuring the levels of soluble HLA-G in the medium at least 84 hours post fertilization; and
transferring the embryo into a compatible human uterus.

53. An IVF program of claim 52 wherein the level of soluble HLA-G is from about 0.150 to about 0.300 OD₄₅₀.

54. An in vitro fertilization (IVF) program comprising:
contacting a human egg with a human sperm to form an fertilized egg;
growing the resulting embryo in vitro in a chemically defined medium;
measuring the levels of soluble HLA-G in the medium at least 96 hours post fertilization; and
transferring the embryo into a compatible human uterus.

55. An IVF program of claim 54 wherein the level of soluble HLA-G is from about 0.150 to about 0.300 OD₄₅₀.
